(19) United States
(12) Patent Application Publication
(10) Pub. No.: US 2017/0073287 A1
(43) Pub. Date: Mar. 16, 2017

(54) PROCESS FOR THE MANUFACTURE OF TERPHENYL COMPOUNDS

(71) Applicant: SOLVAY SPECIALTY POLYMERS USA, LLC, Alpharetta, GA (US)

(72) Inventor: Chantal LOUIS, Alpharetta, GA (US)

(73) Assignee: SOLVAY SPECIALTY POLYMERS USA, LLC, Alpharetta, GA (US)

(21) Appl. No.: 15/125,239

(22) PCT Filed: Mar. 11, 2015
(86) PCT No.: PCT/EP2015/055114

§ 371 Date: Sep. 12, 2016

Related U.S. Application Data
(60) Provisional application No. 61/951,784, filed on Mar. 12, 2014.

(30) Foreign Application Priority Data
May 6, 2014 (EP) 14167124.8

(51) Int. Cl. C07C 37/20

(52) U.S. Cl. CPC C07C 37/20

(57) ABSTRACT

A process for the manufacture of terphenyl compounds comprising reacting at least one compound of general formula (I) with at least one cyclic compound and wherein the process is carried out in the presence of a mixture which contains at least one Lewis acid, optionally, at least one thio compound of formula R—S—R′ and at least one co-catalyst compound selected from the group consisting of C₁-C₁₀ alkanols, C₁-C₁₀ amides, C₁-C₁₀ ethers and C₁-C₁₀ amines; and wherein the ratio of the total molar amount of Lewis acid and co-catalyst compound to the molar amount of compound (C) is equal to or above 5:1 and equal to or below 20:1 and the molar ratio co-catalyst compound to Lewis acid is equal to or above 0.01:1.

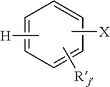

### CROSS-REFERENCE TO RELATED APPLICATIONS

This application claims priority to U.S. provisional application No. 61/951,784 filed on Mar. 12, 2014 and to European application No. 14167124.8 filed May 6, 2014, the whole content of each of these applications being incorporated herein by reference for all purposes.

### FIELD OF INVENTION

The present invention relates to a process for the manufacture of terphenyl compounds, in particular 4,4″-dihydroxy-p-terphenyls.

### BACKGROUND OF THE INVENTION

Dihydroxyterphenyls, in particular 4,4″-dihydroxy-p-terphenyls are very useful starting materials in the manufacturing of polymeric materials, in particular polyarylene ether sulfone (PAES) polymers, which are particularly suitable in more demanding, corrosive, harsh chemical, high-pressure and high-temperature (HP/HT) environments, such as notably in oil and gas downhole applications.

Specifically, 4,4″-dihydroxy-p-terphenyls can be prepared by various ways.

4,4″-dihydroxy-p-terphenyl can notably be synthesized by a Kumada coupling of anisole magnesiumbromide and 1,4-dibromobenzene in the presence of a Pd catalyst such as notably described in Y. K. Han, A. Reiser, Macromolecules, 1998, V 31, P 8789-8793 and A. K. Salunke et al, J. Polym. Sci., Part A: Polymer Chemistry, 2002, Vol. 40, 55-69. However, the use of a homogeneous palladium catalyst and of brominated raw materials presents a high cost.

Alternatively, 4,4″-dihydroxy-p-terphenyl can be obtained by tetrazotization of 4,4″-diaminoterphenyl, thereby forming a tetrazotized product. Replacement of the the diazonium groups by hydroxyl groups in an acid environment results in the forming of 4,4″-dihydroxy-p-terphenyl, as notably described by Charles C. Price and George P. Mueller in J. Am. Chem. Soc., 1944, 66 (4), pp 632-634.

Further, U.S. Pat. No. 5,008,472 discloses a process for preparing 4,4″-dihydroxyterphenyl by sulfonation of the terphenyl moiety thereby resulting in the formation of terphenyl-4,4″-disulfonic acid. Caustic hydrolysis of said terphenyl-4,4″-disulfonic acid, which may be in the form of a dialkali metal salt, provides 4,4″-dihydroxyterphenyl.

The disadvantage of these two routes, as described above, is the extremely high cost of the starting raw material p-terphenyl.

EP 0 343 798 A1 describes the preparation of 4,4″ dihydroxy-p-terphenyl, 4-hydroxybiphenyl and related compounds by condensation of cyclic diones or ketones with phenols and dehydrogenation in the presence of a catalyst, in particular a Pd/C catalyst and a base.

In view of all the above, there is still a current shortfall in the art for an improved process for the manufacture of dihydroxy terphenyl compounds, in particular 4,4″ dihydroxy-p-terphenyl, which can provide dihydroxy terphenyl compounds in high yield, in an efficient manner and having very high purity, and thus suitable to be used as a low cost commercial industrial process.

### DETAILED DESCRIPTION OF THE PRESENT INVENTION

The Applicant has now found that it is possible to advantageously manufacture dihydroxy terphenyl compound, and derivatives thereof, in a very high yield by an easily accessible industrial process, which is advantageously overcoming all the drawbacks of prior art process, as mentioned above.

It is thus an object of the present invention, a process for the manufacture of a terphenyl compound [compound (T), herein after] of formula (T):

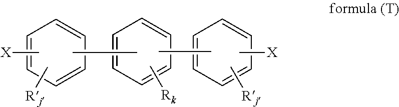

wherein
X is selected from the group consisting of OH, SH, OR₁ and SR₁, wherein each of R₁, equal to or different from each other, is selected from a C₁-C₄ alkyl, C₁-C₄ fluoroalkyl or aryl, X is preferably OH;
each of R and R′, equal to or different from each other, are selected from the group consisting of halogen, alkyl, aryl, ether, thioether, carboxylic acid, ester, amide, imide, sulfonic acid, alkali or alkaline earth metal sulfonate, alkyl sulfonate, phosphonic acid, alkali or alkaline earth metal phosphonate, alkyl phosphonate, amine and quaternary ammonium;
each of j′ and k, equal to or different from each other, are zero or are an integer from 1 to 4
   comprising reacting at least one compound of formula (I):

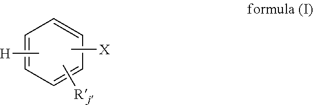

wherein X, R′ and j′, have the meanings given above,
with at least one cyclic compound [compound (C), herein after] complying with those of formulae (II-a), (II-b) (II-c) and (II-d):

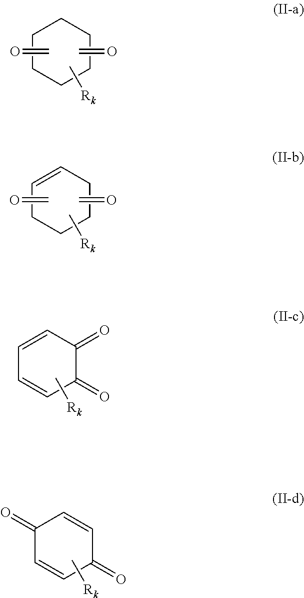

wherein
R and k, have the meanings given above;
   and wherein the process is carried out in the presence of a mixture [mixture (M)] which contains:
at least one Lewis acid,
optionally, at least one thio compound of formula R—S—R′, wherein each of R and R′, equal to or different from each other, are selected from the group consisting of hydrogen, alkyl, aryl, alkyl carboxylic acid.
at least one co-catalyst compound selected from the group consisting of C₁-C₁₀ alkanols, C₁-C₁₀ amides, C₁-C₁₀ ethers and C₁-C₁₀ amines; and wherein the ratio of the total molar amount of Lewis acid and co-catalyst compound to the molar amount of compound (C) is equal to or above 5:1 and equal to or below 20:1 and the molar ratio co-catalyst compound to Lewis acid is equal to or above 0.01:1.

The Applicant has surprisingly found that respecting specific total molar amounts of Lewis acid and co-catalyst compound relative to the molar amount of compound (C), as defined above and whereby the molar ratio of the co-catalyst compound to Lewis acid is also equal to or above 0.01:1 has significantly improved the yield in the manufacturing process of compound (T).

The ratio of the total molar amount of Lewis acid and co-catalyst compound to the molar amount of compound (C) is generally equal to or above 5:1, preferably equal to or above 5.5:1, preferably equal to or above 6:1.

The ratio of the total molar amount of Lewis acid and co-catalyst compound to the molar amount of compound (C) is generally equal to or below 18:1, preferably equal to or below 16:1, preferably equal to or below 14:1.

Very good results have been obtained with a ratio of the total molar amount of Lewis acid and co-catalyst compound to the molar amount of compound (C) of equal to or above 5:1 and equal to or below 14:1.

The molar ratio co-catalyst compound to Lewis acid is in general equal to or above 0.05:1, preferably equal to or above 0.10:1, preferably equal to or above 0.15:1 and more preferably equal to or above 0.3:1.

The molar ratio co-catalyst compound to Lewis acid is in general equal to or below 5:1, preferably equal to or below 3:1, preferably equal to or below 2.5:1.

Very good results have been obtained with a molar ratio co-catalyst compound to Lewis acid of equal to or above 0.1:1 and equal to or below 2.5:1.

In formula (T), the respective phenylene moieties may independently have 1,2-, 1,4- or 1,3-linkages to the other moieties different from R or R′ in the formula (T). Preferably, said phenylene moieties have 1,3- or 1,4-linkages, more preferably they have 1,4-linkage.

Still, in formula (T), j′ and k are preferably at each occurrence zero, that is to say that the phenylene moieties have no other substituents than those enabling linkage in the main chain of the polymer.

Thus, preferred compounds (T) of the present invention are selected from a group consisting of 1,1′:4′,1″-terphenyl-4,4″-diol (i.e. 4,4″-dihydroxy-p-terphenyl), 1,1′:3′,1″-terphenyl-4,4″-diol, 1,1′:2′,1″-terphenyl-4,4″-diol. Most preferred compound (T) is 1,1′:4′,1″-terphenyl-4,4″-diol.

Within the context of the present invention the mention “at least one compound of formula (I)” is intended to denote one or more than one compound of formula (I). Mixtures of compounds of formula (I) can advantageously be used for the purposes of the invention.

In the rest of the text, the expressions “compound of formula (I)” are understood, for the purposes of the present invention, both in the plural and the singular, that is to say that in the process of the present invention one or more than one compound of formula (I) may be reacted.

Among preferred compounds of formula (I), as detailed above, suitable for being used in the process of the present invention, mention may be notably made of phenol, anisole, thiophenol, thioanisole, alkylphenol. Most preferred compound of formula (I) is phenol.

Within the context of the present invention the mention “at least one cyclic compound [compound (C), herein after]” is intended to denote one or more than one compound (C). Mixtures of compound (C) can be advantageously used for the purposes of the invention.

In the rest of the text, the expressions “compound (C)” are understood, for the purposes of the present invention, both in the plural and the singular, that is to say that in the process of the present invention one or more than one compound (C) may be reacted.

More preferred compounds (C) are those complying with following formulae shown below:

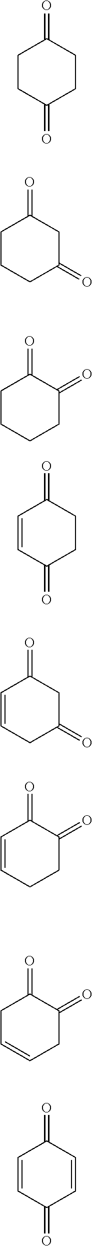

Particularly preferred compounds (C) are 1,4-cyclohexanedione, 1,3-cyclohexanedione, 1,2-cyclohexanedione or a mixture thereof. Most preferred compound (C) is 1,4-cyclohexanedione.

Within the context of the present invention the mention “at least one Lewis acid” is intended to denote one or more than one Lewis acid. Mixtures of Lewis acid can advantageously be used for the purposes of the invention.

In the rest of the text, the expressions “Lewis acid” are understood, for the purposes of the present invention, both in the plural and the singular, that is to say that in the process of the present invention one or more than one Lewis acid may be present.

For the purpose of the present invention, the term “Lewis acid”, denotes organometallic and, especially inorganic Lewis acids.

In a preferred embodiment, the inorganic Lewis acid may be selected from a group of compounds including, but not being limited to, inorganic halides and inorganic oxides. Inorganic halides are preferred Lewis acids.

Preferably, the inorganic halides have the formula MXₙ wherein M is a component selected from the Group IIB, IIIA, IIIB, IVA, IVB, VA, VB, VIB or VIII Elements of the Periodic Table or their mixtures, X is a halogen, n is the atomic ratio of halogen to M and varies from 1-7. Preferably, M is selected from the Group Ib, IIb, IIIA, IVA, VIB or VIII.

M is preferably Fe, Zn, Cr, Ni, Cu, and Al. X can be considered to be a single type of halogen even though it should be understood that X could refer to a mixtures of halogens such that MXₙ could be, for example, AlClF₂.

Preferably, X is a chloride, a bromide or a fluoride anion. More preferably, X is chloride or bromide. Most preferably X is a chloride. Suitable inorganic chloride Lewis acids are including, but not limited to, AlCl₃, SnCl₄, FeCl₃, NiCl₂, FeCl₂, FeCl₃, CuCl₂, NbCl₅, TiCl₄, and ZnCl₂. More preferred inorganic chloride Lewis acids are AlCl₃, FeCl₃ and ZnCl₂.

In another preferred embodiment, the organometallic Lewis acid may be selected from a group of compounds including, but not being limited to alkoxides, sulfonic acid salts and carboxylic acid salts.

Among preferred thio compound of formula R—S—R′, if any present, suitable for being used in the process of the present invention, mention may be notably made of H₂S, alkyl thiols, such as notably 1-methylthiol, 1-hexanethiol, 1-octanethiol, mercaptoacetic acid. Alkyl thiols are especially preferred.

Good results were obtained with 1-octanethiol.

Within the context of the present invention the mention “at least one co-catalyst compound” is intended to denote one or more than one co-catalyst compound. Mixtures of co-catalyst compounds can advantageously be used for the purposes of the invention.

In the rest of the text, the expressions “co-catalyst compound” are understood, for the purposes of the present invention, both in the plural and the singular, that is to say that in the process of the present invention one or more than one co-catalyst compound may be present.

Among preferred C₁-C₁₀ alkanols suitable for being used in the process of the present invention, mention may be notably made of methanol, ethanol and isopropanol. Methanol is especially preferred.

Among preferred C₁-C₁₀ amides suitable for being used in the process of the present invention, mention may be notably made of N-Methyl-2-pyrrolidinone (NMP), N,N-dimethylformamide and N,N-dimethylacetamide. NMP is especially preferred.

Among preferred C₁-C₁₀ ethers suitable for being used in the process of the present invention, mention may be notably made of diethyl ether, dioxane and tetrahydrofuran (THF).

Among preferred C₁-C₁₀ amines suitable for being used in the process of the present invention, mention may be notably made of triethylamine, diethylamine, diisopropylethylamine and pyridine.

Preferred co-catalyst compounds are C₁-C₁₀ alkanols, C₁-C₁₀ amides and mixture thereof.

Good results were obtained when the co-catalyst compound is methanol and/or NMP.

In the process according to the invention, the molar ratio of the compound of formula (I), as described above, to compound (C), as described above, is advantageously above 2:1, preferably above 5:1, more preferably above 10:1 and most preferably above 12:1.

Upper limit of the compound of formula (I) to compound (C) is not particularly critical and will be selected by the skilled in the art in view of economical reasons.

In one embodiment of the process according to the invention, the molar ratio of the compound of formula (I), as defined above, to compound (C), as described above, is advantageously below 35:1, preferably below 30:1, more preferably below 25:1 and most preferably below 20:1.

In the process according to the invention, the molar ratio of the compound of formula (I), as described above, to compound (C), as described above, is advantageously between 2:1 to 35:1, preferably between 5:1 to 30:1, more preferably between 10:1 to 25:1 and most preferably between 12:1 to 20:1.

The compound of formula (I), being advantageously used in excess of compound (C), may take over the role of being a solvent at the same time.

If desired, the process of the present invention is carried out in the presence of an additional solvent.

For the purpose of the present invention, the term “additional solvent” is understood to denote a solvent different from the reactants and the products of the process of the present invention.

Suitable additional solvents for use in the process according to the invention include, not limited to, toluene, xylene, 1,2,4-trimethylbenzene, 1,2,3-trimethylbenzene, methylene chloride, 1,2-dichlorobenzene, 1,3-dichlorobenzene, 1,4-dichlorobenzene, 1,2,4-trichlorobenzene, 1,2,3-trichlorobenzene and mixtures thereof.

The process according to the present invention is preferably carried out at each step of the process at a temperature of below 320° C., more preferably of below 280° C., still more preferably of below 270° C. and most preferably of below 265° C. On the other hand, the process according to the present invention is preferably carried out at a temperature of above 20° C., more preferably of above 25° C., still more preferably of above 30° C. and most preferably of above 40° C.

The process according to the present invention is advantageously pursued while taking care to avoid the presence of any reactive gases in the reactor. These reactive gases may be notably oxygen, water and carbon dioxide. O₂ and water are the most reactive and should therefore be avoided.

In a particular embodiment, the reactor should be evacuated under pressure or under vacuum and filled with an inert gas containing less than 20 ppm of reactive gases, and in particular less than 10 ppm of O₂ and less than 10 ppm water prior to adding the compound (C) to the reaction mixture. Then, the reactor should be put under a constant purge of said inert gas until the end of the reaction. The inert gas is any gas that is not reactive under normal circumstances. It may be chosen from nitrogen, argon or helium. The inert gas contains preferably less than 10 ppm oxygen, 20 ppm water and 20 ppm carbon dioxide.

According to certain embodiments, the process according to the present invention is preferably carried out at a pressure of below 10 atm, more preferably of below 7 atm, still more preferably of below 5 atm and most preferably of below 2 atm. On the other hand, the process according to the present invention is preferably carried out at a temperature of above 0.5 atm, more preferably of above 0.6 atm, still more preferably of above 0.7 atm and most preferably of above 0.8 atm. Excellent results were obtained when the process according to the present invention was carried out at atmospheric pressure.

According to other embodiments, the process according to the present invention is preferably carried out at a pressure of below 60 atm, more preferably of below 55 atm, still more preferably of below 50 atm and most preferably of below 45 atm. On the other hand, the process according to the present invention is preferably carried out at a temperature of above 0.5 atm, more preferably of above 0.6 atm, still more preferably of above 0.7 atm and most preferably of above 0.8 atm.

In a particular embodiment, the process according to the present invention for the manufacture of a compound (T), as detailed above, comprises the following steps a. to e.:
a. at least one compound (C), as detailed above, is added to a reaction medium at a temperature T1 wherein said reaction medium is comprising, preferably consisting, of at least one compound of formula (I), as detailed above, and a mixture (M), as detailed above,
b. the reaction medium is maintained to at least one temperature T2 for a reaction time t_{b} of at least 1 hour
c. optionally, at least one base, at least one dehydrogenation catalyst and an aqueous solution are added to the reaction medium or optionally, at least one base catalyst is added to the reaction medium
d. optionally, the reaction medium is maintained to at least one temperature T3 for a reaction time t_{d} of at least 0.5 hour
e. the compound (T) is isolated from the reaction medium

In step a., the temperature T1 is preferably of below 100° C., more preferably of below 95° C., still more preferably of below 90° C. and most preferably of below 80° C. On the other hand, the temperature T1 is preferably of above 30° C., more preferably of above 35° C., still more preferably of above 40° C. Good results were obtained when T1 was from 40° C. to 90° C.

In one specific embodiment of the present invention, prior to step a., the at least one compound of formula (I), as detailed above and the mixture (M), as detailed above, are added at the same time to the reactor which is then advantageously evacuated under pressure or under vacuum and filled with an inert gas containing less than 20 ppm of reactive gases, and in particular less than 10 ppm of O₂ and 10 ppm water prior to adding of the compound (C) to the reaction mixture in step a.

In another specific embodiment of the present invention, prior to step a., the at least one compound of formula (I), as detailed above, is first added to the reactor which is then advantageously evacuated under pressure or under vacuum and filled with an inert gas containing less than 20 ppm of reactive gases, and in particular less than 10 ppm of O₂ and the mixture (M), as detailed above is then added to the reactor at a temperature T1.

If desired, the at least one Lewis acid, as detailed above, optionally the thio compound of formula R—S—R′, as detailed above, and the at least one co-catalyst compound, as detailed above, of the mixture (M) may be added simultaneous or sequentially to the reactor at a temperature T1.

In step a., the at least one compound (C), as detailed above, is preferably added very slowly, typically, over a time of from 10 minutes to 300 minutes, preferably from 60 minutes to 250 minutes and more preferably from 90 to 150 minutes.

If desired, step a. of the process of the present invention according to this particular embodiment may be omitted and the at least one compound of formula (I), as detailed above, the mixture (M), as detailed above, and the at least one compound (C), as detailed above, are added at the same time to the reactor. It is understood that the reactor have advantageously been evacuated under pressure or under vacuum and filled with an inert gas containing less than 20 ppm of reactive gases, and in particular less than 10 ppm of O₂ and 10 ppm water prior to addition of the at least one compound of formula (I), as detailed above, the mixture (M), as detailed above, and the at least one compound (C), as detailed above.

After step a., in step b., the reaction medium is preferably maintained at a T2. The temperature T2 in step b. is preferably of below 220° C., more preferably of below 200° C., still more preferably of below 180° C. and most preferably of below 160° C. On the other hand, the temperature T2 is preferably of above 20° C., more preferably of above 25° C., more preferably of above 30° C., still more preferably of above 40° C. Good results were obtained when T2 was comprised between 40 and 160° C. Excellent results were also obtained when the temperature T2 of step b. was varied with time, so that different temperatures T2 were maintained during said step b.

In general, the reaction time t_{b} in step b. is of at least 2 hours, preferably of at least 4 hours, preferably of at least 5 hours, preferably of at least 6 hours, preferably of at least 7 hours, preferably of at least 8 hours, preferably of at least 9 hours.

Upper limit of the the reaction time t_{b} is not particularly critical and will be selected by the skilled in the art.

Among suitable bases, if any added in step c., mention can be made of NaOH, KOH, NaOCH₃, KOCH₃, NaOtBu, KOtBu.

Among suitable dehydrogenation catalysts, if any added in step c., mention can be made of palladium on carbon (i.e. Pd—C) and palladium on alumina.

Among suitable base catalysts, if any added in step c., mention can be made of Na phenate, K phenate.

The aqueous solution, if any added in step c., may be water or aqueous solutions of C₁-C₄ alkanols. The aqueous solution is preferably water.

If desired, in step c., an optional hydrogen acceptor such as notably α-methylstyrene may further be added to the reaction medium.

When step c. is carried out, the at least one base, the at least one dehydrogenation catalyst and the aqueous solution, in particular water, or the at least one base catalyst are preferably added very slowly, typically, over a time of from 5 minutes to 10 hours, depending on the cooling capacity of the reaction medium.

In general, at the end of step c., thus after adding said at least one base, said at least one dehydrogenation catalyst and the aqueous solution, in particular water, or said at least one base catalyst, the temperature is raised to at least one temperature T3 and preferably maintained in step d. to at least one temperature T3.

The temperature T3 in step d. is preferably of below 320° C., more preferably of below 300° C., still more preferably of below 280° C. and most preferably of below 270° C. On the other hand, the temperature T3 is preferably of above 160° C., more preferably of above 180° C., still more preferably of above 200° C. and most preferably of above 220° C. Excellent results were also obtained when the temperature T3 of step d. was varied with time, so that different temperatures T3 were maintained during said step d.

In general, the reaction time t_{d} in step d. is of at least 1 hour, preferably of at least 1.5 hours, preferably of at least 2 hours, preferably of at least 2.5 hours, preferably of at least 3 hours, preferably of at least 3.5 hours.

Upper limit of the the reaction time t_{b} is not particularly critical and will be selected by the skilled in the art.

According to a preferred embodiment, the process was carried out during step d. under autogeneous pressure with the aim to keep the water in the mixture.

In step e., the compound (T) may be isolated from the reaction medium by precipitation, crystallization or extraction, which can be carried out according to standard practice of the skilled in the art.

Good results were obtained when the compound (T) and in particular 1,1′:4′,1″-terphenyl-4,4″-diol was isolated by precipitation by adding a combination of (i) a water immiscible solvent such as toluene, xylene, 1,2,4-trimethylbenzene, 1,2,3-trimethylbenzene, methylene chloride, 1,2-dichlorobenzene, 1,3-dichlorobenzene, 1,2,4-trichlorobenzene, 1,2,3-trichlorobenzene, methylisobutyl ketone, (ii) an acid or a base and (iii) water, by liquid-liquid extraction or by distillation under vacuum.

Suitable acids may include, but not limited to, concentrated hydrochloric acid (HCl), acetic acid, citric acid, salicylic acid.

Suitable bases may include, but not limited to, ethylenediaminetetraacetic acid bis sodium salt (EDTA), K₂CO₃, Na₂CO₃.

According to one embodiment, the process of the present invention is carried out in one pot. The term “one pot” when referred to a reaction is generally intended to denote any reaction where a reactant is subjected to successive chemical reactions in just one reactor, thereby avoiding a lengthy separation process and purification of the intermediate chemical compounds.

Thus, steps a. to d. may all be carried out in one reactor

According to an alternative embodiment, the process of the present invention is carried out in at least two or more pots, preferably in two pots.

In an alternative embodiment, after step c., thus by adding a step (c′), the reaction medium may be transferred to a second reactor, such as notable a pressure reactor.

In this particular alternative embodiment, in step (c′), the second reactor is typically evacuated under pressure or under vacuum and filled with an inert gas containing less than 20 ppm of reactive gases, and in particular less than 10 ppm of O₂. Said inert gas may be chosen from nitrogen, argon or helium. The inert gas contains preferably less than 10 ppm oxygen, 20 ppm water and 20 ppm carbon dioxide.

The temperature of the reaction medium in said second reactor in step (c′), is preferably increased to at least one temperature T3′. During step (c′), the reaction medium is preferably maintained to at least one temperature T3′. The temperature T3′ is preferably of below 320° C., more preferably of below 300° C., still more preferably of below 280° C. and most preferably of below 270° C. On the other hand, the temperature T3′ is preferably of above 160° C., more preferably of above 180° C., still more preferably of above 200° C. and most preferably of above 220° C. Excellent results were also obtained when the process was carried out during step (c′) at different temperatures T3′.

According to a preferred embodiment, the process was carried out during step (c′) under autogeneous pressure with the aim to keep the water in the mixture.

Should the disclosure of any patents, patent applications, and publications which are incorporated herein by reference conflict with the description of the present application to the extent that it may render a term unclear, the present description shall take precedence.

The invention will be now described in more details with reference to the following examples, whose purpose is merely illustrative and not intended to limit the scope of the invention.

### Raw Materials

Concentrated hydrochloric acid (37%) was procured from Fisher

1-octanethiol, 98% was procured from Aldrich

Aluminum chloride, anhydrous, 98%, was procured from Acros

Ferric chloride, anhydrous, 97%, was procured from Aldrich

Zinc chloride, anhydrous, 98%, was procured from Aldrich

1,4-cyclohexanedione, 98%, was procured from Aldrich

Phenol, 98%+, was procured from Aldrich

Palladium on charcoal, 5 wt %, was procured from Aldrich

α-methylstyrene, 99%, stabilized by 15 ppm t-butylcatechol, was procured from Aldrich

N-methylpyrrolidinone, electronic grade, was procured from ISP and dried on molecular sieves type 4A to <50 ppm water Methanol, HPLC garde, 99.9% was procured from Aldrich.

### Manufacturing of 1,1′:4′,1″-terphenyl-4,4″-diol

### Example 1

In a 500 mL 4-neck glass reactor fitted with a stirrer, a N₂ inlet tube, a Claisen adapter with a thermocouple plunging in the reaction medium, and a Barrett trap with a condenser were introduced 188.12 g of phenol (2.00 mol). The reactor was sealed and under nitrogen was heated up to 50° C. The molten phenol was then held under nitrogen for 30 minutes to evacuate air. Via an addition funnel, 0.0503 g of 1-octanethiol (0.003 mol), 12.39 of N-methylpyrrolidinone (NMP) (0.125 mol) were added to the phenol.

Via a PTFE-flex line, 101.40 g of ferric chloride (0.625 mol) were added while keeping the reaction mixture under nitrogen.

Using a powder dispenser, 14.02 g of 1,4-cyclohexanedione (0.125 mol) were added to the mixture over a period of 2 hours. The mixture was held at 50° C. for 7 hours, then heated to 100° C. The mixture was still held at 100° C. for 7 hours then cooled down to room temperature. The reaction mixture was cooled down to room temperature under nitrogen. Excess of phenol was distilled off the reaction mixture.

1,1′:4′,1″-terphenyl-4,4″-diol was extracted from the reaction mixture upon addition of methylisobutylketone, concentrated HCl and water.

The yield is shown in Table 1.

### Example 2

Example 2 is prepared in the same way as example 1, except that other molar amounts are used for the reagents phenol, 1-octanethiol, methylpyrrolidinone (NMP), ferric chloride and 1,4-cyclohexanedione, as shown in Table 1.

### Example 3

In a 500 mL 4-neck glass round-bottom fitted with a stirrer, a N₂ inlet tube, a Claisen adapter with a thermocouple plunging in the reaction medium, and a Barrett trap with a condenser were introduced 188.18 g of phenol (2.00 mol), 0.503 g of 1-octanethiol (0.003 mol), 127.81 g of zinc chloride (0.937 mol) and 22.38 g of methanol (0.698 mol). The flask was sealed and under nitrogen was heated up to 70° C. Using a powder dispenser, 14.02 g of 1,4-cyclohexanedione (0.125 mol) were added to the mixture over a period of 2 hours. The mixture was held at 70° C. for 15 hours.

Under nitrogen the reaction mixture was then heated to 120° C. and held at this temperature for 7 h. The reaction mixture was cooled down to room temperature under nitrogen.

1,1′:4′,1″-terphenyl-4,4″-diol was extracted from the reaction mixture upon addition of methylisobutylketone, concentrated HCl and water.

The corresponding yield is shown in Table 1.

### Comparative Example 4

In a 500 mL 4-neck glass reactor fitted with a stirrer, a N₂ inlet tube, a Claisen adapter with a thermocouple plunging in the reaction medium, and a Barrett trap with a condenser were introduced 18811 g of phenol (2.00 mol). The reactor was sealed and under nitrogen was heated up to 50° C. The molten phenol was then held under nitrogen for 30 minutes to evacuate air. Via an addition funnel, 0.504 g octanethiol (0.003 mol) 18.59 g NMP (0.187 mol) were added to the phenol, then 50.00 g aluminum chloride (0.375 mol) were added via flex PTFE line to the reaction mixture.

Using a powder dispenser, 14.017 g of 1,4-cyclohexanedione (0.125 mol) were added to the mixture over a period of 2 hours. The mixture was held at 50° C. for 7 hours, then heated up to 100° C. and held at 100° C. for 7 hours.

The reaction mixture was cooled down to room temperature under nitrogen. 1,1′:4′,1″-terphenyl-4,4″-diol was extracted from the reaction mixture upon addition of methylisobutylketone, concentrated HCl and water.

The corresponding yield is shown in Table 1.

### Example 5

In a 500 mL 4-neck glass reactor fitted with a stirrer, a N₂ inlet tube, a Claisen adapter with a thermocouple plunging in the reaction medium, and a Barrett trap with a condenser were introduced 120.40 g of phenol (1.28 mol). The reactor was sealed and under nitrogen was heated up to 50° C. The molten phenol was then held under nitrogen for 30 minutes to evacuate air. Via an addition funnel, 0.316 g octanethiol (0.002 mol) 14.32 g of methanol (0.447 mol) were added to the phenol, then 53.34 g aluminum chloride (0.40 mol) were added via flex PTFE line to the reaction mixture.

Using a powder dispenser, 8.97 g of 1,4-cyclohexanedione (0.080 mol) were added to the mixture over a period of 2 hours. The mixture was held at 50° C. for 2 hours then heated up to 70° C. for 5 hours.

Providing cooling with an ice batch, 50.40 g of sodium hydroxide, 25.20 g water and 0.602 g of palladium on carbon were added slowly to the mixture.

Under nitrogen, the reaction mixture was then heated to 190° C. At 190° C., the outlet of the reactor was connected to a vacuum line (50 mbar) for the reaction mixture was held under vacuum at 190° C. for 4 hours, during which phenol and water were collected in the Barrett trap. The vacuum was disconnected and the reaction mixture was cooled down to room temperature under nitrogen. 1,1′:4′,1″-terphenyl-4,4″-diol was extracted from the reaction mixture upon addition of 130.34 g of concentrated HCl and 130 g water.

The corresponding yield is shown in Table 1.

### Example 6

In a 1000 mL 4-neck glass reactor fitted with a stirrer, a N₂ inlet tube, a Claisen adapter with a thermocouple plunging in the reaction medium, and a Barrett trap with a condenser were introduced successively: 500.00 g phenol (5.31 mol), 1.336 g 1-octanethiol (0.009 mol), 110.72 g aluminum chloride (0.830 mol) and 59.43 g methanol (1.855 mol). The reactor was sealed and under nitrogen and was heated up to 50° C. Using a powder dispenser, 37.230 g of 1,4-cyclohexanedione (0.332 mol) were added to the mixture over a period of 2 hours. The mixture was held at 50° C. for 7 hours.

Providing cooling with an ice batch, 132.80 g of sodium hydroxide, 370.00 g water and 1.700 g of palladium on carbon and 78.47 g α-methylstyrene (0.664 mol) were added slowly to the mixture.

The mixture was then transferred to a Parr reactor, the air in the reactor was evacuated by 3 pressure/depressure cycles with nitrogen, and the temperature was increased to 260° C. under autogeneous pressure and held at temperature for 6 hours. When the mixture had cooled down, 1,1′:4′,1″-terphenyl-4,4″-diol was extracted from the reaction mixture upon addition of methylisobutylketone, acetic acid and water.

The corresponding yield is shown in Table 1.

TABLE 1

| Examples | 1 | 2 | 3 | C4 | 5 | 6 |
|---|---|---|---|---|---|---|
| Molar amounts | | | | | | |
| Phenol | 2.00 | 5.31 | 2.00 | 2.00 | 1.28 | 5.31 |
| 1,4-cyclohexanedione | 0.125 | 0.332 | 0.125 | 0.125 | 0.080 | 0.332 |
| Lewis Acid | | | | | | |
| AlCl₃ | | | | 0.375 | 0.400 | 0.830 |
| FeCl₃ | 0.625 | 2.49 | | | | |
| ZnCl₂ | | | 0.937 | | | |
| 1-octanethiol | 0.003 | 0.009 | 0.003 | 0.003 | 0.002 | 0.009 |
| co-catalyst compound | | | | | | |
| MeOH | | | 0.699 | | 0.447 | 1.855 |
| N-methylpyrrolidinone | 0.125 | 1.01 | | 0.19 | | |
| Ratio of total molar | 6.0 | 10.5 | 13.1 | 4.5 | 10.6 | 8.1 |
| amount of Lewis Acid and | | | | | | |
| co-catalyst compound to | | | | | | |
| molar amount 1,4- | | | | | | |
| cyclohexanedione | | | | | | |
| Ratio of molar amount of | 1.00 | 0.41 | 0.75 | 0.51 | 1.12 | 2.24 |
| co-catalyst to molar | | | | | | |
| amount of Lewis Acid | | | | | | |
| Yield % | 34.0 | 37.8 | 28.9 | 8.3 | 38.0 | 40.8 |

1-15: (canceled)

16. A process for manufacturing a terphenyl compound (T) of formula: 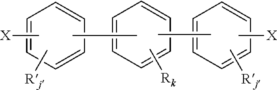
wherein:
X is selected from the group consisting of OH, SH, OR₁, and SR₁, wherein each of R₁, equal to or different from each other, is selected from a C₁-C₄ alkyl, C₁-C₄ fluoroalkyl or aryl;
each of R and R′, equal to or different from each other, are selected from the group consisting of halogen, alkyl, aryl, ether, thioether, carboxylic acid, ester, amide, imide, sulfonic acid, alkali or alkaline earth metal sulfonate, alkyl sulfonate, phosphonic acid, alkali or alkaline earth metal phosphonate, alkyl phosphonate, amine, and quaternary ammonium;
each of j′ and k, equal to or different from each other, are zero or are an integer from 1 to 4;
the process comprising reacting at least one compound of formula (I): 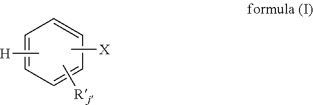
wherein X, R′ and j′, have the meanings given above,
with at least one cyclic compound (C) of formulae (II-a), (II-b), (II-c), (II-d), and mixtures thereof: 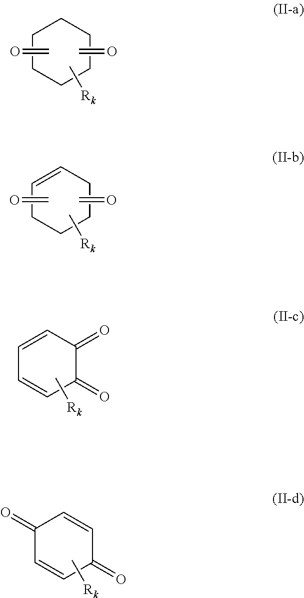
wherein:
R and k, have the meanings given above;
and wherein the process is carried out in the presence of a mixture (M) comprising:
at least one Lewis acid;
optionally, at least one thio compound of formula R—S—R′, wherein each of R and R′, equal to or different from each other, are selected from the group consisting of hydrogen, alkyl, aryl, alkyl carboxylic acid;
at least one co-catalyst compound selected from the group consisting of C₁-C₁₀ alkanols, C₁-C₁₀ amides, C₁-C₁₀ ethers and C₁-C₁₀ amines, wherein the ratio of the total molar amount of Lewis acid and co-catalyst compound to the molar amount of compound (C) is equal to or above 5:1 and equal to or below 20:1 and the molar ratio co-catalyst compound to Lewis acid is equal to or above 0.01:1.

17. The process according to claim 16, wherein the molar ratio co-catalyst compound to Lewis acid is equal to or below 5:1.

18. The process according to claim 16, wherein the compound of formula (I) is selected from the group consisting of phenol, anisole, thiophenol, thioanisole, and alkylphenol.

19. The process according to claim 16, wherein compound (C) is of the formulae:

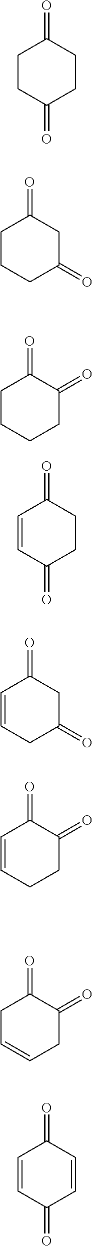

20. The process according to claim 16, wherein the Lewis acid is an inorganic Lewis acid selected from an inorganic halide or inorganic oxide.

21. The process according to claim 20, wherein the inorganic halide has formula MXₙ wherein M is a component selected from the Group IIB, IIIA, IIIB, IVA, IVB, VA, VB, VIB or VIII Elements of the Periodic Table or their mixtures, X is a halogen, n is the atomic ratio of halogen to M and varies from 1-7.

22. The process according to claim 16, wherein the co-catalyst compound is selected from C₁-C₁₀ alkanols, C₁-C₁₀ amides or mixtures thereof.

23. The process according to claim 16, wherein the process is carried out in the presence of an additional solvent, wherein the additional solvent is selected from the group consisting of toluene, xylene, 1,2,4-trimethylbenzene, 1,2,3-trimethylbenzene, methylene chloride, 1,2-dichlorobenzene, 1,3-dichlorobenzene, 1,4-dichlorobenzene, 1,2,4-trichlorobenzene, 1,2,3-trichlorobenzene and mixtures thereof.

24. The process according to claim 16, wherein the compound (T) is selected from a group consisting of 1,1′:4′,1″-terphenyl-4,4″-diol and 1,1′:3′,1″-terphenyl-4,4″-diol.

25. The process according to claim 16, wherein the process comprises:
adding the at least one cyclic compound (C) to a reaction medium at a temperature T1, wherein said reaction medium comprises the at least one compound of formula (I) and the mixture (M), and wherein the temperature T1 is below 100° C. and above 30° C.;
maintaining the reaction medium at a temperature T2 for a reaction time t_{b} of at least 1 hour, wherein the temperature T2 is at least one temperature below 220° C. and above 20° C.;
optionally adding at least one base, at least one dehydrogenation catalyst, and an aqueous solution to the reaction medium, or optionally adding at least one base catalyst to the reaction medium;
optionally maintaining the reaction medium at a temperature T3 for a reaction time t_{d} of at least 0.5 hour; and
isolating the compound (T) from the reaction medium.

26. The process according to claim 25, wherein the at least one base, the at least one dehydrogenation catalyst, and the aqueous solution are added to the reaction medium, and the at least one base is selected from NaOH, KOH, NaOCH₃, KOCH₃, NaOtBu, KOtBu, and mixtures thereof, and the at least one dehydrogenation catalyst is selected from palladium on carbon or palladium on alumina.

27. The process according to claim 25, wherein the temperature T3 is below 320° C. and above 160° C.

28. The process according to claim 25, wherein the process is carried out in one reactor, except for isolating the compound (T) from the reaction medium.

29. The process according to claim 25, wherein before optionally maintaining the reaction medium at the temperature T3 for the reaction time t_{d}, the reaction medium is transferred to a second reactor.

30. The process according to claim 29, wherein the temperature of the reaction medium is raised and maintained at temperature T3′, wherein temperature T3′ is at least one temperature below 320° C. and above 160° C.

31. The process according to claim 22, wherein the co-catalyst compound is methanol, NMP, or mixtures thereof.

32. The process according to claim 16, wherein X is OH.
